(19) United States
(12) Patent Application Publication
(10) Pub. No.: US 2015/0297913 A1
(43) Pub. Date: Oct. 22, 2015

(54) Flexible Light Treatment Pad

(71) Applicant: Meditech International Inc., Toronto (CA)

(72) Inventors: Alexander L. KNAUS, Richmond Hill (CA); Eli HACCO, Toronto (CA)

(73) Assignee: MEDITECH INTERNATIONAL INC., Toronto (CA)

(21) Appl. No.: 14/690,768

(22) Filed: Apr. 20, 2015

Related U.S. Application Data
(60) Provisional application No. 61/982,101, filed on Apr. 21, 2014.

(51) Int. Cl. A61N 5/06; B32B 38/00; F21V 33/00; B32B 37/18; H05K 1/18; F21V 29/70

(52) U.S. Cl. CPC A61N 5/0616; H05K 1/189; F21V 29/70; F21V 33/0068; B32B 37/185; B32B 38/0004; B32B 38/0012; H05K 2201/10106; H05K 2201/10174; F21Y 2101/02

(57) ABSTRACT

The present invention provides a flexible light treatment pad comprising one or more light source modules interconnected in a continuous electrical web and disposed on a flexible circuit board located on a first portion of the light treatment head. The light treatment pad further includes a control module located on a second portion of the light treatment, the second portion being physically separated and spaced apart from the first portion, the control module for providing a control signal to the one or more light source modules. Further, said light source module and said control module are encased in a flexible moulding layer for allowing flexion of the light treatment pad in numerous curvatures.

### CROSS-REFERENCE TO RELATED APPLICATIONS

This application claims priority to U.S. Provisional Patent Application No. 61/982,101 filed on Apr. 21, 2014; the entire contents of which is incorporated herein by reference.

### TECHNICAL FIELD

The following relates generally to the field of light treatment therapy and more specifically to a flexible light treatment pad for such therapy.

### BACKGROUND

The use of light to injuries such as sport injuries and sprains as well as chronic conditions such as arthritis, sciatica, and chronic slow healing wounds or sores, is well known.

The principle of all these light treatments is the application of light radiating in the area of the injury. It is found that in order to be effective, the light source should be in close contact with the skin. The light source is usually an array or panel of light emitting diodes, or in some cases low level laser. The treatment typically becomes more effective over longer periods. The light sources may, for example, be left in contact with the skin for up to sixty minutes or more. This enables deep penetration of the light rays into the tissues, and has been found to be efficacious in many instances.

Treatment heads are used to support the light sources and direct the energy to the treatment area. The design of suitable heads requires consideration of the supply of electrical energy to the light sources, the control of those light sources and of course the topography of the area to be treated. Moreover, dissipation of the heat generated by the sources is a major consideration to avoid injury to the patient. A number of different treatments have been proposed. For example, in U.S. patent application Ser. No. 13/355,162, one or more light source modules are interconnected by a hinge assembly. The hinge assembly comprises a plurality of hinge joints and each hinge joint includes a wiring channel. A cover is provided to cover the joints. This arrangement provides a robust construction that allows independent movement of the adjacent modules to enhance the flexibility of the head. The relative motion between the modules of the treatment head is however limited to a single axis by the hinge apparatus, so that treatment of surfaces with compound curvature may be less than satisfactory.

Some injuries require a large area to be treated, which leads to a relatively large head that may have to be moved during treatment. In such cases, where mechanical components are used, the mass of the head may impose an uncomfortable load on the patient and require significant effort to move.

It is therefore an object of the present invention to obviate or mitigate at least one of the above presented disadvantages.

In one aspect, there is provided a flexible light treatment pad comprising one or more light source modules interconnected in a continuous electrical web and disposed on a flexible circuit board located on a first portion of the light treatment head. The light treatment pad further includes a control module located on a second portion of the light treatment head. The circuit board and control module are encased in a flexible body of material for allowing flexion of the light treatment pad in a plurality of degrees of curvatures. The flexible body allows flexure about multiple axes to accommodate compound contours of a patient's body.

Preferably, the second portion is physically separated and spaced apart from the first portion within the body and as a further preference the control module provides a control signal to the one or more light source modules. Preferably, during the moulding process, the body is formed with a first layer on one side of the circuit board and a second layer on the opposite side of the circuit board. As a further preference, the light sources are arranged on the one side of the circuit board and the first layer is thinner than the second layer. In one example, there is utilized a 1:2 ratio of the thickness between the first layer and second layer.

### DISCLOSURE OF THE INVENTION

Embodiments of the invention will now be described by way of example only with reference to the accompanying drawings.

### BRIEF DESCRIPTION OF THE DRAWINGS

FIG. 1 is a perspective view of a light treatment pad and controller positioned to treat an injury to the wrist of a patient;

FIG. 2 is a perspective view, similar to FIG. 1 of a light treatment pad positioned to treat an injury to the elbow of a patient;

FIG. 3 is a block diagram of the functional components of the controller and light treatment pad of FIGS. 1 and 2;

FIG. 4 is a block diagram of the functional components of a multiple arrays of single wavelength LED treatment head;

FIG. 5 is a block diagram of a power control module of a arrays of bi-colour LED array;

FIG. 6 is a plan view of one side of the light treatment pad;

FIG. 7 is a view similar to FIG. 6 of the opposite side of the light treatment pad;

FIG. 8 is a side view of the light treatment pad of FIGS. 6 and 7

FIG. 9 is a front elevation of an alternative embodiment of treatment pad;

FIG. 10 is an exploded view of the embodiment of FIG. 9

FIG. 11 is a section on the line X1-X1 of FIG. 9.

### DETAILED DESCRIPTION OF THE DRAWINGS

The present invention provides a flexible light treatment pad. The light treatment pad is configured for attachment and/or positioning on a patient for receiving light treatment therapy. The light treatment pad is preferably manufactured by compression molding a body that encompasses a flexible circuit board within.

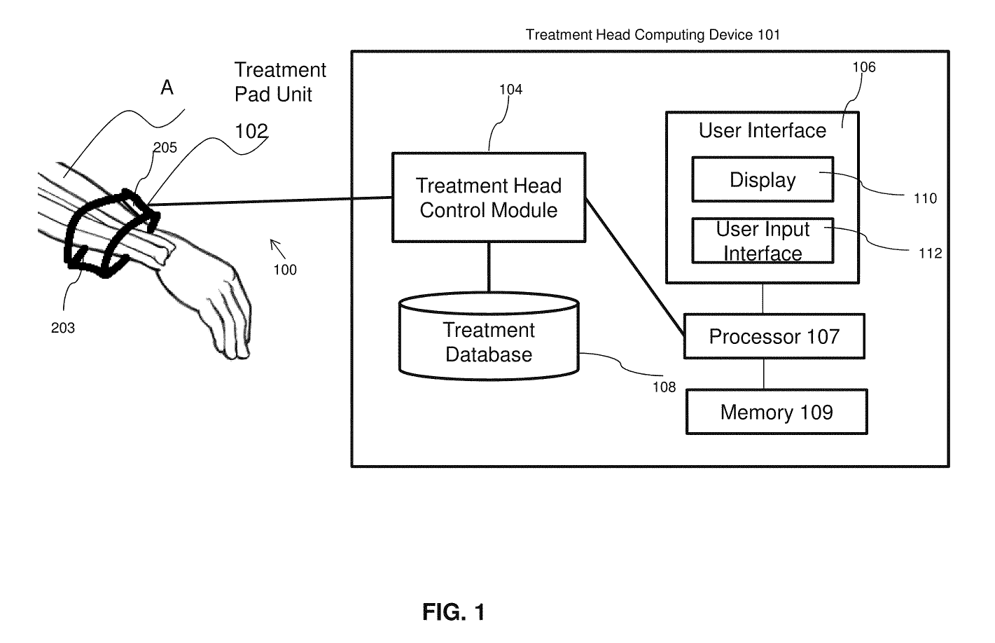

An example of a light treatment system 100 is provided in FIGS. 1 through 7. The light treatment system 100 comprises a treatment pad 102, and a treatment head computing device 101. The treatment head computing device 101 is preferably external to the treatment pad 102 and in communication therewith. The treatment head computing device 101 comprises a treatment head control module 104, a user interface 106, a processor 107, a treatment database 108, and a memory 109. The user interface 106 further comprises a display 110, and a user input interface 112.

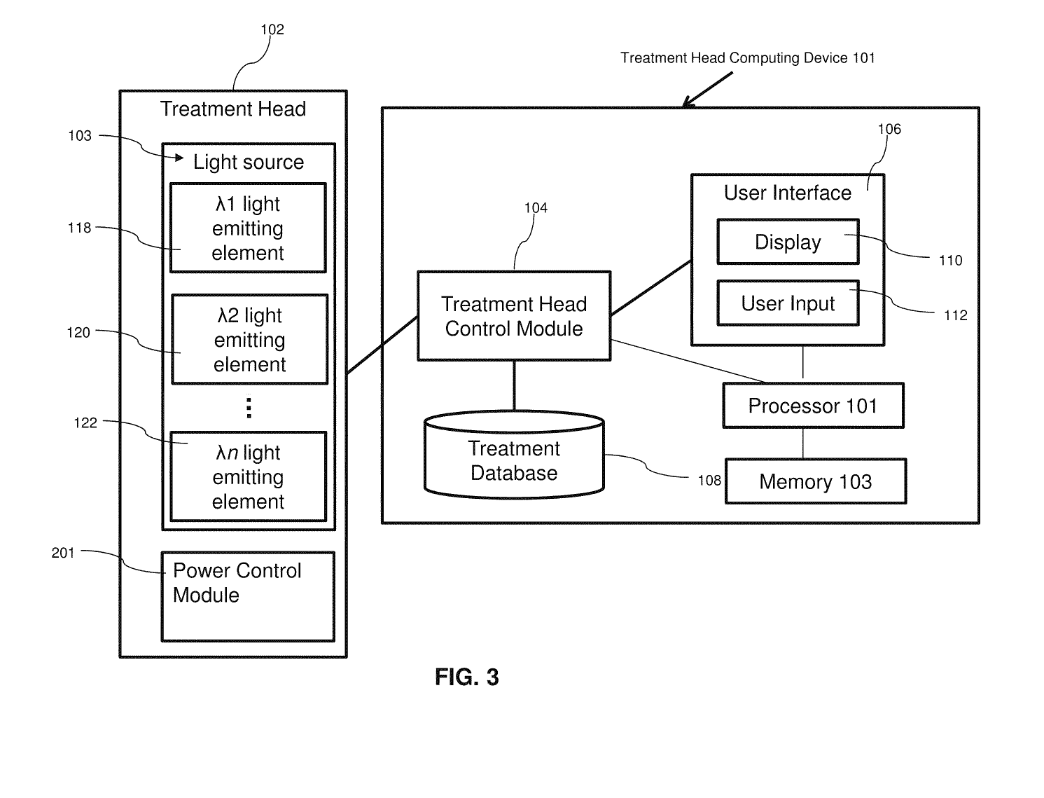

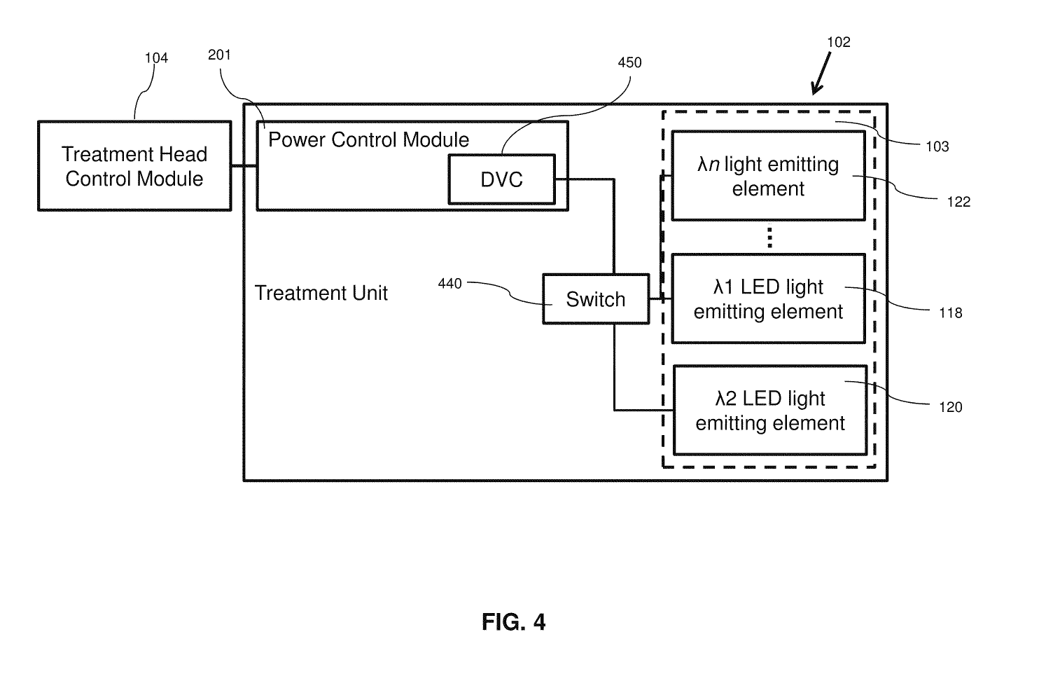

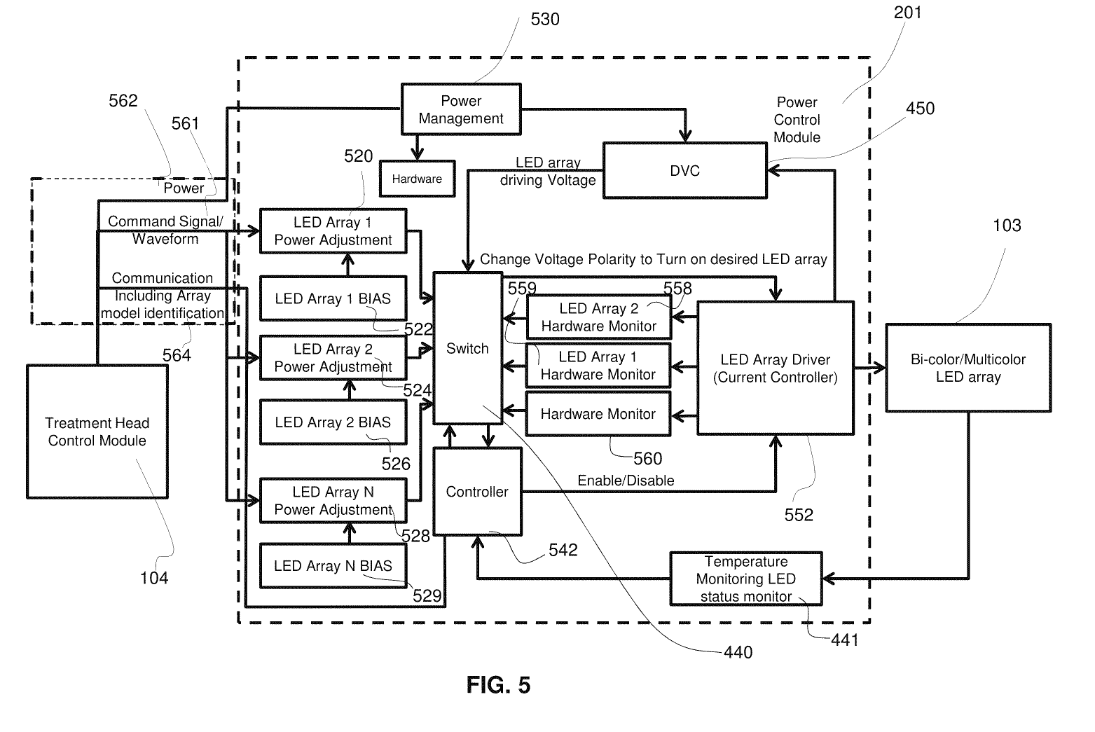

The treatment pad 102 is substantially flexible for being positioned on and for conforming to the shape and curvatures of the patient's body that is receiving the light treatment. The treatment pad 102 comprises a power control module 201 and one or more light source modules 103, as shown in FIGS. 3 to 5. The light source modules 103 are interconnected electrically on a flexible circuit board 211 and disposed on one side 105 of the board 211. The modules 103 are constrained to a first portion 203 of the treatment pad 102. The power control module 201 is mounted on a second portion 205 of the board 211 and controls power delivered to one or more of the light source modules 103. In one embodiment, the light modules 103 may be an array of LED's emitting a single wavelength, as shown in FIG. 3, or arrays of bi-color LED's as shown in FIGS. 4 and 5. In each case, a suitable power control module 201 controls power to the arrays in response to signals from the control module 104, as will be described more fully below.

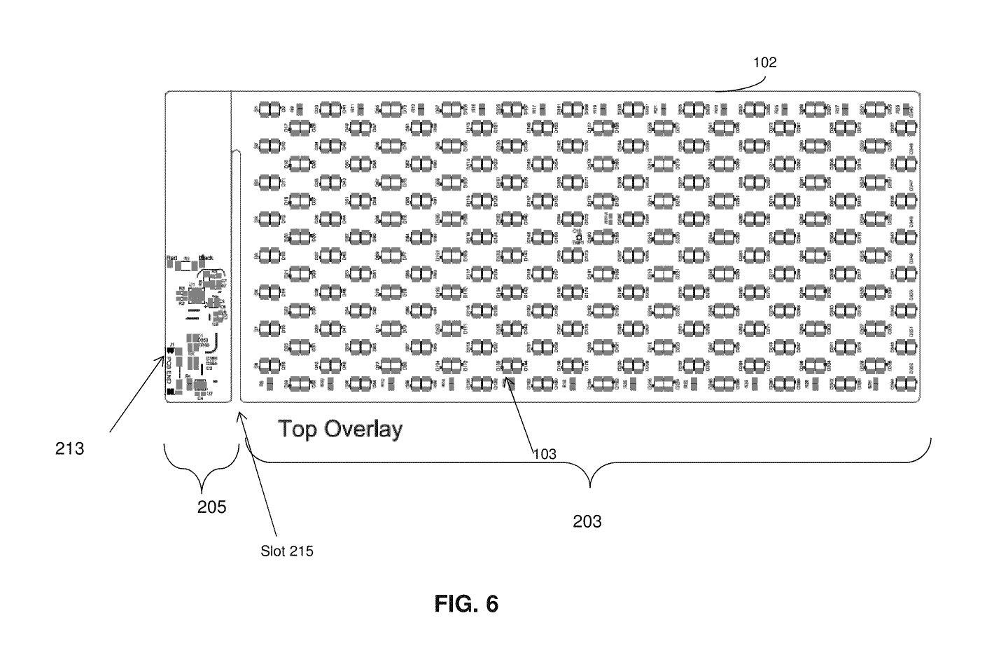

A connector interface 213 (FIG. 6) is provided on the second portion 205 of the board 211 for communicatively coupling the treatment pad 102 to the treatment head computing device 101 and for allowing communication of control and/or monitoring data there between. The connector interface 213 is typically a USB interface for electrically coupling the treatment pad 102 to the treatment head computing device 101, although other forms of connectors can be used, such as a wireless interface for communication (e.g. Bluetooth, ultra-wideband (UWB), ZigBee, or Wi-Fi). Further alternatively, the connector interface 213 can connect to the treatment head computing device 101 across a network such as the Internet.

The treatment pad 102 is configured to receive one or more control signals from the treatment head computing device 101 for causing the emission of light from one or more of the light source modules 103 onto a desired area of the patient's body at one or more predefined wavelength(s).

Each light source module 103 comprises one or more light emitting elements (indicated at 118, 120 and 122 in FIGS. 3, 4 and 5). The light emitting elements comprises light emitting diodes (LED), or, the light emitting elements may comprise LASER or a combination of LED and LASER. It will be appreciated that the light emitting elements will be chosen to provide the wavelength and type of radiation required to produce the desired therapeutic effect.

In one embodiment shown in FIG. 3, arrays of light emitting elements 118, 120, 122 of different wavelengths λ1, λ2, λ3 respectively, are used to create a multi-colour treatment head 102. For example, the treatment pad unit 102 may comprise an array of LEDs, a first group of which comprise light emitting element 118 and emit at 660 nm while a second group comprising light emitting element 120 and emit at 840 nm. The LEDs may, for example, be arrayed such that the LEDs 118 of the first wavelength λ1 are substantially evenly distributed throughout the treatment pad unit and the LEDs 120 of the second wavelength λ2 are also substantially evenly distributed throughout the treatment pad unit and interposed between the LED's 118.

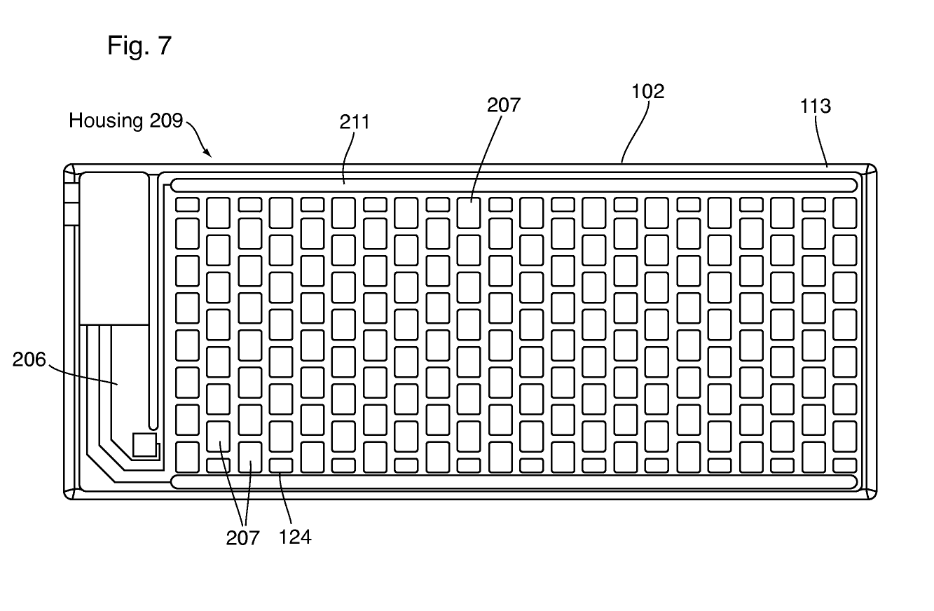

Referring to FIGS. 6 and 7, each light emitting element 118, 120, 122, has a pair of conductors, 124, each of which is electrically connected to a respective connector pad 207. The connector pads 207 are disposed on the opposite side 206 of the board 104 to the modules 103, so as to be directed in the opposite direction to the modules 103. Preferably, the connector pads 207 occupy a larger surface area than the light emitting elements (e.g. 118 and 120) with a minimal gap between each of the connector pads 207. The major portion of side 206 of the circuit board 211 is thus occupied with the connector pads 207 in order to achieve a maximum surface area for effective heat dissipation and act as a heat sink body. As shown in FIG. 7, the connectors 207 are square and arranged linearly to coincide with the array of the light emitting elements 118. Where a different array is required, alternative shapes may be provided, such as hexagonal, triangular or rectangular to give a nested arrangement covering one of the major surface of the board 211.

The shape and configuration of the connector pads 207, which advantageously are made of copper for good heat and electrical conductivity, is selected to maximize the surface area of the connector pads 207 relative to the surface area of the circuit board 211 while forming the electrical conductive traces on the circuit board 211. The flexible circuit board 211 is preferably made from any one or more of the following materials: polyimide copper clad with or without adhesive, polyester copper clad or any flexible film or metal clad.

Referring again to FIG. 6, the first and the second portions 203 and 205 of the board 211 are mechanically isolated from each other by a discontinuity or slot 215. The slot 215 extends across the board 211 from one edge and terminates prior to the opposite edge to provide a bridge 212 between the two portions 203 and 205. The bridge 212 provides an area of the board 211 that allows electrical connectivity between the first and the second portion 203 and 205. The extent of the slot 215 will vary depending on the flexibility required and the connections to be made between the two portions. In general, the slot will extend as far as practical without jeopardising the structural integrity of the board 211. In a typical embodiment, the slot 215 will extend across approximately 75% of the width of the board, with ranges of between 70% and 85% of the board being practical. In an example of a board with an overall width of 92.33 mm, the slot extended 74.53 mm from one edge. The width of the slot was 1.35 mm and it was located 19.68 mm from one end of the board. The overall length of the board was 224.03 mm.

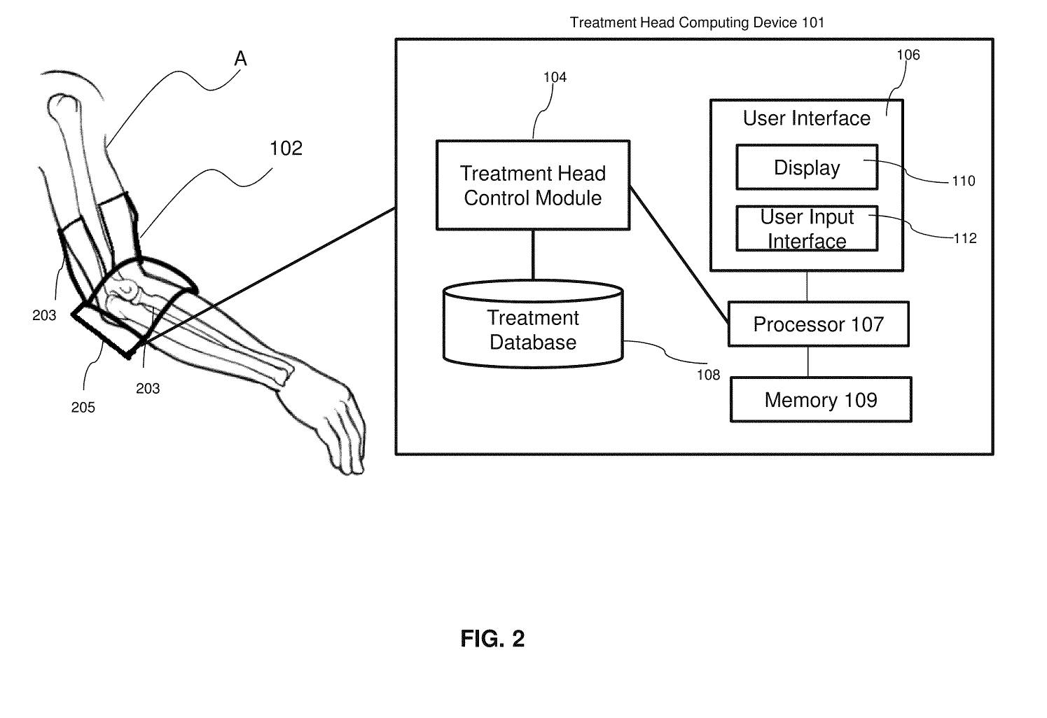

The second portion 205 supports or incorporates the generally inflexible or rigid circuitry components (e.g. drivers, communication interfaces, control circuitry, switches) of the treatment pad unit 102. The slot 215 provides a partition for physically separating or isolating the flexible from the inflexible circuitry components on the treatment pad 102. In this manner, the first portion 203 that needs to be in contact (e.g. placed on a surface for receiving treatment) with the patient for providing treatment is able to flex to contour to the surface area to be treated while the second portion 205 that does not need to be in direct contact with the patient's skin and so does not need to flex, is not subjected to the strains imparted by such flexure. The first portion 203 is thus able to flex along a number of different axes to conform to the shape of the patient's skin, and conform to highly contoured areas of the patient, such as the wrist shown in FIG. 1 or the elbow shown in FIG. 2.

The flexible circuit board 211 is encased within a flexible housing 209 made from an insulating material 209 that is optically transparent to the wavelength of the radiation emitted by the modules 103. The insulating material 209 is preferably a high consistency silicone rubber, such as that available from Wacker Chemicals AG—SILPURAN® 8020/40. Generally, the moulding layers forming the housing 209 can be selected from the group consisting of: silicone, thermoplastic, polyurethane, and thermoplastic elastomer (TPE).

In the embodiment of FIGS. 3 to 8, the housing 209 is preferably formed using a compression molding technique, as will be described in more detail below, and has a pair of layers 220, 221 separated by the flexible circuit board 211 (e.g. see FIG. 7). The layer 220 overlying the light emitting modules 103 is optically transparent and has a minimal thickness to enhance coupling of the modules 103 to the skin of the patient. The layer 221 overlying the connectors 207 may be of the same material or a different material and may be thicker than the layer 220. The thickness of the layer 221 is selected to ensure heat dissipation from the connector pads 207 whilst protecting the circuit elements. In one example, the layer 220 is 2.4 mm thick and the layer 221 is 3.2 mm thick. However, other variations of thickness and size of the layers 220 and 221 can be envisaged.

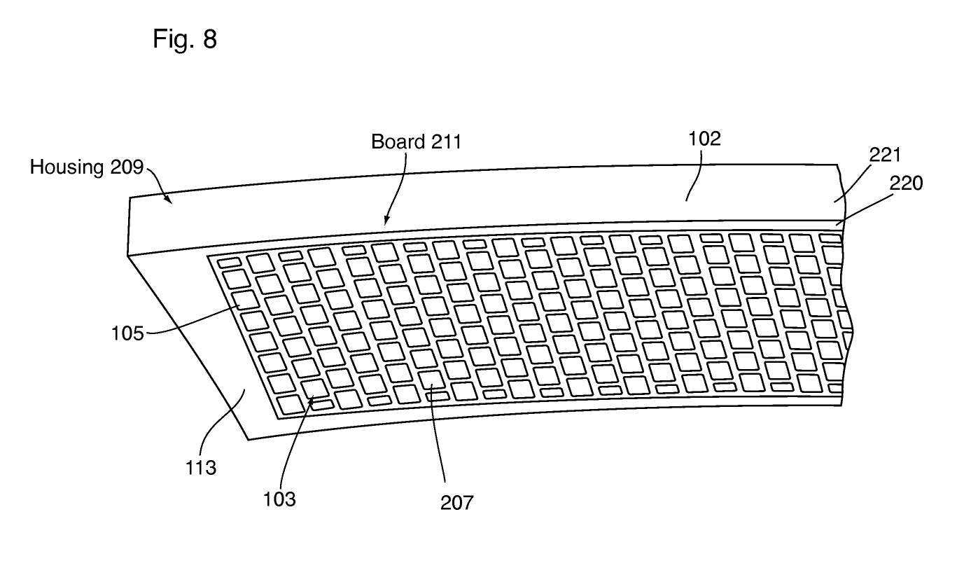

The layers 220, 221 extend beyond the edges of the circuit board 211 and are joined to one another to provide a continuous margin about the periphery of the board 211, as indicated at 113 in FIGS. 7 and 8, so that a self-contained flexible treatment pad 102 is provided.

Accordingly, the treatment pad unit 102 can flexed upwards and/ or downwards in the first portion 203 along an axis parallel to the bridge 202. The treatment pad unit 102 can also be flexed upwards and/or downwards in the first portion 203 and is only limited in the direction where the first portion 203 is directly adjacent to the bridge 202. The separation of the inflexible components in the second portion 205 from the flexible components in the first portion 203 by the slot 215 allows a high degree of pliability and flexion in the overall treatment pad unit 102 as the second portion 205 occupies a substantially smaller surface area than the first portion 203. Additionally, by separating the flexible from the inflexible circuit components, this provides a stress relief to the relatively rigid components mounted on the treatment pad unit 102 during flexure and bending for conforming to various body contours and reduces the likelihood of damage to the treatment pad unit 102 components.

The second inflexible portion 205 can either rest on the patient's body adjacent to the first portion 203 (as shown in FIG. 1) or extend outwardly from the treatment surface (e.g. as shown in FIG. 2). Where appropriate, the first portion 203 can for example be folded over itself to form a loop and the two ends of the first portion 203 connected together by a fastener (e.g. a strap or other connector).

As the treatment pad 102 is configured to curve around limbs, the available curvature is preferably around 40 mm diameter minimum (in the side of the LEDs adjacent to the patient's skin) and 160 mm backwards in the opposite side to allow for example, conforming to the inside curve of the neck to the shoulder. The spacing between the light emitting elements 118, 120 and another set of light emitting elements corresponds to the average power density required (total surface area of the pad divided by the power of each element). Preferably, this distance is minimized while considering the power output needs discussed herein.

The strap may be a fabric, plastic or comprise a stretchable portion configured for coupling to opposite ends of the treatment pad unit 102 for connecting the treatment pad unit 102 across portions of the patient's body.

As noted above, the circuit board 211 is encased in two Silicone layers which are applied to respective upper and lower surfaces of the circuit board 211 of the treatment pad unit 102 to form the housing 209. The housing 209 is formed by compression molding of the silicone layers surrounding the circuit components on circuit board 211. In this arrangement, the substantially flexible circuit board 211 is mounted between two sheets of silicone, and heat and pressure is applied to the silicone layers such that as it cures, it seals the edges of the moulding layer to form the housing 209.

In one example, a board having a length of 225 mm and width of 95 mm was placed in a mold having dimensions 259×120 mm.

The housing may be formed in the following manner. A sheet of high consistency silicone rubber is placed in a mold cavity and then a flex board populated with light emitting devices and circuitry is disposed thereon. A second layer of silicone was then placed on top of the circuit boards and the first (lower) layer of silicone. All three layers are then preferably volcanized together by compressing the layers (e.g. layers 220, 221 and circuit board 211) within the mold and raising the temperature.

The following chart provides exemplary moulding parameters used for forming the layers:

Moulding Temperatures: 240 F., 285 F. and 320 F.
Moulding Materials: HCR (high consistency silicone rubber)
Moulding Processes: compression molding and/or rolling to form the
layers
Material Thickness (upper layer) ~ 3/32″
Material Thickness (lower layer) ~⅛″

Alternatively, the flexible moulding layers (e.g. layers 220, 221) forming the flexible housing 209 can be formed by for example, injection molding, sandwich molding, casting or other such methods can be envisaged.

Where compression molding is used, the process for compression molding and forming the layers for the housing 209 comprises: 1. Rolling the material (e.g. flexible moulding layers 220, 221 forming the flexible housing 209) to pre-defined respective thicknesses (e.g. approximately 3/32″); 2. Cutting or pre-forming the material to the size of the mold. 3. Layering of material in the mold, and 4. Curing the material. By controlling the size and thickness of the material shear forces imposed on the devices and flex board by movement of material inside the mold, such as might result from expansion during curing, may be mitigated. The volume of the material will be as close as possible to the volume of the final product (e.g. flexible housing 209). The layer on top of the LEDs may be thinner than the layer on the bottom (e.g. layer 221) to compensate for the volume of the LEDs and components in the circuit board 211 and maintain the minimum distance of the LED's from the treatment area.

In the embodiment of FIGS 1-8, the connector/ Surface Mount Technology (SMT) components within the flexible housing 209 and between the layers 220, 221 may be located within or covered by a secondary housing such as for example, any one of: a molded plastic cover, a metal bracket or housing, an epoxy or any other kind of cover. The secondary housing helps to hold down the connector and protect it (and the circuit 211 components) from damage during encapsulation or in use. Alternately, for the array of LEDs and/or circuit board 211 components having a wire/cable perturbing (instead of a connector), the wire/cable perturbing may be formed as a loop, or similar slack portion of cable, to prevent damage by pulling (or stressing) the cable in the finished encapsulated or molded flexible housing 209.

The cable may be tied down to the rigid portion of the flexible board assembly (e.g. the second inflexible portion 205), as may be necessary.

Other exemplary materials used for the flexible housing 209 can include different kinds of rubber, thermoplastic elastomers, PVC and/or polyurethane. Preferably, the moulding parameters are selected to minimize the thickness of the housing and to maximise light transmission so as to impart more light energy into the body. A thinner housing also improves flexibility without compromising the strength of the treatment pad 102 and protects the circuitry inside as well with the housing 209.

It has been found surprisingly that despite encasing the light source modules 103 in silicone, the heat from the light emitting elements (e.g. LED) can be dissipated from the added surface area of copper in the flexible circuit board 211, even when encapsulated in rubber.

In a further embodiment, an element formed from shape memory alloy may be embedded within the housing 209 for retaining the shape of the contours of the patient's body being treated. The shape memory alloy is disposed as strips within the housing and will conform as the housing 209 is flexed. The shape memory alloy strips maintain the housing 209 in the flexed position, but can of course be reconfigured for different applications. Shape memory alloy strips along each edge of the housing 209 allow the pad to be configured to the normal needs of the user.

In a further variation, the flexible circuit board 211 is perforated between sets of the light emitting elements (e.g. 118 and 120) to aid in flexibility of the overall circuit board 211 and also to allow adhesion between the layers forming the housing 209 during the molding process.

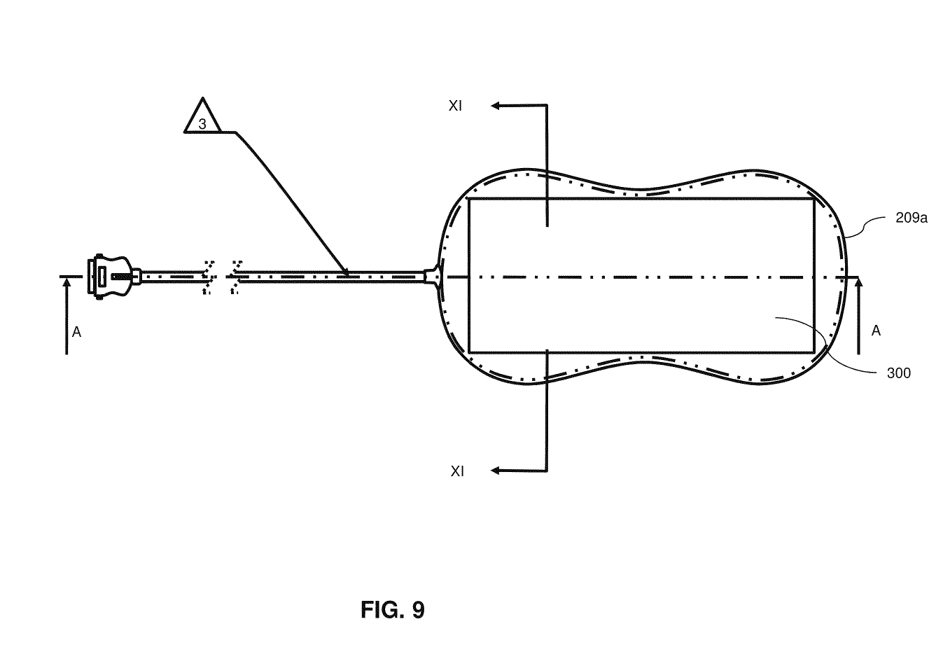

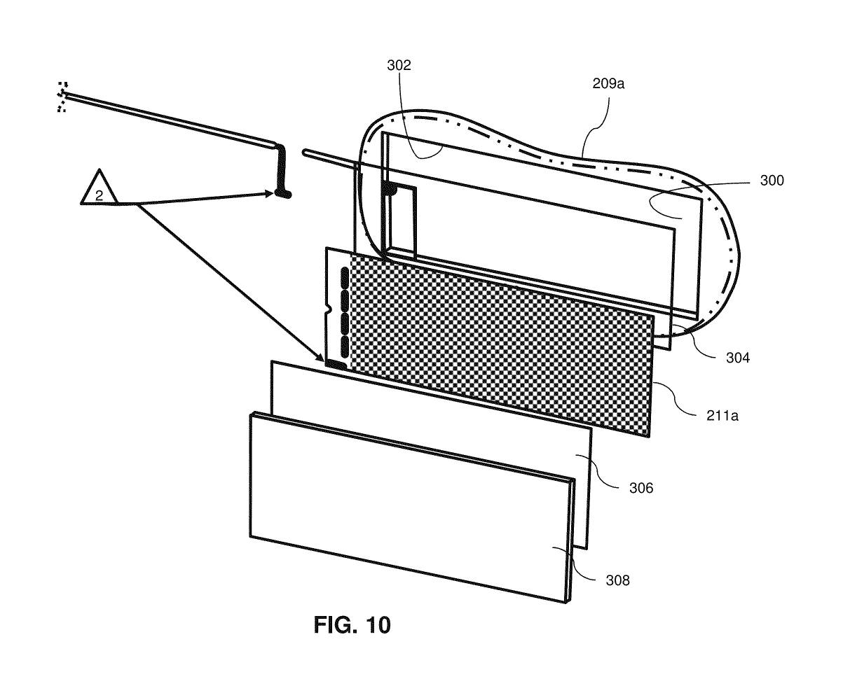

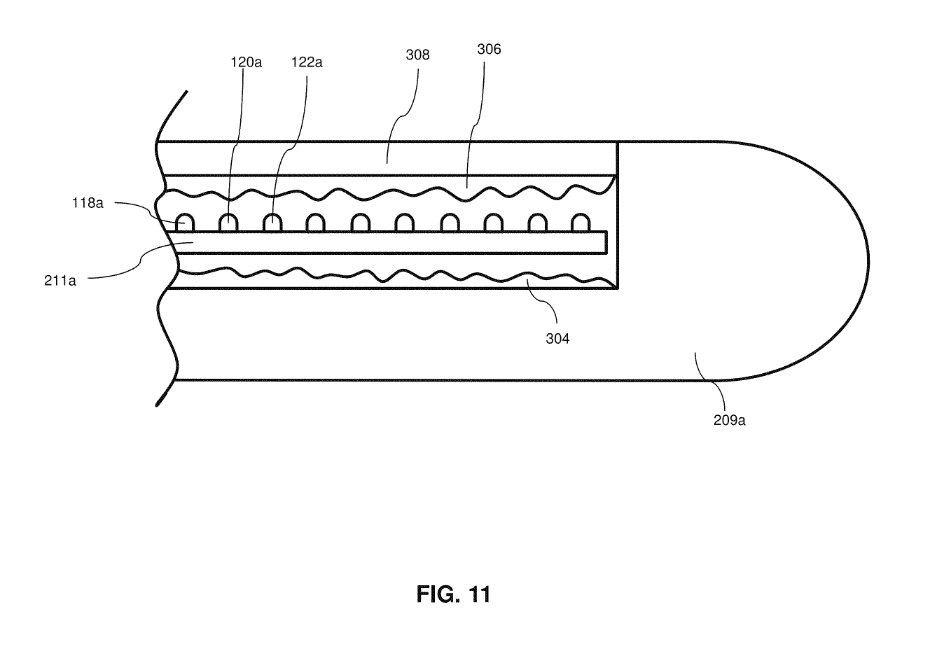

A further embodiment of treatment pad unit is shown in FIGS. 9 to 11 in which like components will be identified by like numerals with a suffix “a” added for clarity. The housing 209a is pre-molded to have a pocket 300 with a peripheral upstanding edge 302. The pocket 300 is dimensioned to accommodate the circuit board 211 a and locate it during subsequent molding operations. A layer 304 of non-woven material is located in the pocket 300 to act as a barrier to separate the circuit board 211a from the housing 209a, and a similar layer 306 of non-woven material is placed across the face of the LED's 118a, 120a, 122a. The non-woven material is a relatively thin layer having a nominal thickness of 0.30 mm. A suitable material is Typar 3301 available from TYPAR Geosynthetics.

The assembled housing and circuit board is over-molded with optical quality silicon 308 that fuses to the housing to encapsulate the board within the housing. It has been found that the provision of the layers 304, 306 provides a barrier between the board and the housing that is effective to reduce the loads placed on the electrical components when the housing is flexed during use. It has also been found that selected components may advantageously be embedded in epoxy on the board to further strengthen the connection of the board to the components during flexure. The provision of the layer 306 does not impede the transmission of light significantly. The upstanding peripheral edge 302 locates the board within the pocket during the molding process and reduces the stresses induced on the board and the components mounted on the board, notably the LED's, both during the over molding and subsequently in use. To reduce the potential damage to the components during manufacture, it has been found advantageous to allow an increased cooling time in the mold before the mold is opened. The increased cooling time results in a lower temperature of the silicon at the time the mold is opened so it is more stable and less likely to pull on the components. A temperature of 220 degrees F. has been found to be suitable for enhanced stability whilst maintaining a reasonable production time.

Referring to FIG. 6, the light source modules 103 are substantially evenly distributed and spaced on the first portion 203. However, other arrangements may be envisaged where the array of light source modules can be linear, staggered, or other arrangements as envisaged by a person skilled in the art. In the embodiment of FIG. 6, the light source modules 103 are positioned on the first portion 203 in a continuous and repetitive pattern throughout the portion.

As noted above, the light source 103 may comprise at least one light emitting element 118 operable to emit at a first wavelength and at least one other light emitting element 120 operable to emit at a second and distinct wavelength. As shown in FIG. 3, a switch 440 is provided to selectively cause the light emitting elements 118 and 120 to emit. For example, the switch 440 may enable neither light emitting element 118, 120 to emit, both light emitting elements 118, 120 to emit simultaneously, or to select between the light emitting elements 118 and 120.

As will be appreciated, the light source 103 may further comprise additional light emitting elements which are switchable by switch 440 and enable the treatment pad unit 102 to emit at additional, or alternate, wavelengths. For example, the light source 103 may comprise three, four, five, or more light emitting elements of different wavelengths. It will also be appreciated that one or more of the light emitting elements may emit at a plurality of wavelengths, for example, a light emitting element may emit substantially white light.

Referring again to FIGS. 1 and 2, the treatment head control module 104 controls the operation of the treatment pad unit 102 and is in communication with each of the treatment pad unit 102, the user interface 106, and the treatment database 108. The treatment head control module 104 is coupled to a memory 109 for storing computer executable instructions and a processor 107 for executing the computer executable instructions stored in the memory 109.

The treatment head control module 104 controls parameters associated with the light source 103 of the treatment pad unit 102. Specifically, the treatment head control module 104 is operable to control various parameters of the light source 103 including, for example, the intensity of emitted light, the duration of light emission, the number of cycles of treatment to be applied to a particular area of the patient, and the wavelength of light emitted onto the patient.

The treatment head control module 104 further comprises, or is linked to, a power source which powers the light source module(s) 103. In the example embodiment provided with reference to FIG. 4, the power source is incorporated into the treatment head control module 104. The treatment pad unit 102 may otherwise, or in addition, include an on-board power source such as a battery to power the light source module(s) 103.

As outlined above, referring to the treatment head computing device 101, the treatment head control module 104 is in communication with the user interface 106. The user interface 106 can obtain instructions from an operator of the light treatment system 100 via a user input 112 and provide information to an operator via an output, for example, a display 110. The user interface 106 may otherwise, or in addition, include a speaker, one or more indicator lights, a microphone, or various other input and output devices known in the art.

The user interface 106 enables a user to control the light source 103 on the treatment pad unit 102 and to receive data from the treatment pad unit 102 via the treatment head control module 104. This data may include, for example, the operational status of the treatment head 102 (i.e. to determine whether the treatment pad unit 102 is in an operating condition), the operational parameters of the treatment pad unit 102 (i.e. the wavelength(s) and waveform at which the light source 103 is emitting), the temperature of the treatment pad unit 102, and other information relevant to an operator of the light treatment system 100.

The treatment head control module 104 is in communication with a treatment database 108, which is operable to store various treatment protocols. The treatment database 108 may also store patient information including a patient identifier, patient health information, treatment history, injury diagnosis, prescriptions, and other relevant information. Treatment protocols can be selected by an operator based on a patient's prescription of a pre-existing treatment protocol or based on a customized treatment protocol. A treatment protocol includes a combination of treatment steps performed in a treatment session with a patient. An operator of the light treatment system 100 may select an appropriate treatment protocol from the treatment database 108 via the user input 112 of the user interface 106 and view treatment instructions, progress, and other information via the display 110 of the user interface 106. The treatment head control module 104 may select a treatment protocol from the treatment database 108 based on patient identifier stored in a patient database (not shown) and linked to a treatment protocol.

New treatment protocols may be entered into and stored in the treatment database 108 via the user interface 106. Existing treatment protocols may also be linked with a patient identifier via the user interface 106. In one example, the treatment head control module 104 is operable to obtain additional treatment protocols, for example, via a network connection (not shown) and store these in the treatment database 108. These additional treatment protocols may then be linked with selected patient identifiers or selected by an operator of the light treatment system 100 for use during a treatment session.

The diagrams of FIGS. 1 and 2 show exemplary applications of the treatment head 102. In FIG. 1, a treatment head 102 can be located above an elbow joint of a patient's arm A to treat the area above the joint, whereas the treatment head 102 is located below the elbow joint of the patient's arm A in FIG. 2 to treat the area below the joint. Light treatment may be used to treat the elbow joint itself, treat tendonitis in the triceps, or treat osteoarthritis in the joint itself, and the location of the head is selected to promote adequate exposure of the area being treated. It will be appreciated that the treatment head 102 may be used to treat various other conditions and various other areas of a patient's body.

A treatment head 102 comprising a light source 103 is shown in FIG. 3. The light source 103 comprises light emitting elements 118 and 120 which emit at a first wavelength and a second wavelength, respectively. The light source 103 may further include additional light emitting elements 122 that emits at various other wavelengths.

The light source module 103 comprises one or more light emitting elements 118, 120 disposed along the treatment surface of the light treatment head 102. The light emitting elements 118, 120 may comprise LEDs including organic LEDs (OLEDs), fibre optics coupled to a light guide, LASER emitters, or various other light emitting structures and combinations thereof known in the art. It will be appreciated that any light emitting element which provides the energy and intensity of light to achieve the desired therapeutic effect may be used. It will also be appreciated that the light emitting elements 118, 120 may be chosen based on additional properties including their heat generating characteristics of the light source 103, the physical size of the light emitting elements 118, 120, the spectral width of a light emitting element, or the electrical efficiency of a light emitting element 118, 120, and based on other design considerations that would be apparent to a person familiar with light treatment systems.

In use, the light emitting elements 118, 120 are placed in contact with, or in close proximity to, a patient's skin in the area being treated. This placement maximizes penetration of the light emitted by the light source 103 and improves distribution of the light into the area of the patient being treated to maximize the therapeutic effect of the treatment.

Typically, the wavelength and intensity of light emitted by such light emitting elements 118, 120 are relevant to the therapeutic effect. The light emitting elements 118, 120 may emit in the visible range, in the near-infrared, or in the infrared range. The light emitting elements 118, 119 may otherwise, or in addition, emit in the ultra-violet range, for example, to sanitize a portion of a patient's skin, for example, in the vicinity of a wound.

Turning now to FIG. 4, a simplified block diagram of a power control module 201 for controlling a bi-colour LED treatment unit 102 is provided. The treatment head control module 104 is shown in communication with the power control module 201 on the treatment head 102. The power control module 201 is operable to provide power to, and control, one or more light emitting elements, or arrays of light emitting elements. The power control module 201 may drive a first light emitting element operable to emit at a first wavelength 118, a second light emitting element operable to emit at a second wavelength 120. The power control module 201 may further be operable to drive up to n additional light emitting elements 122.

Referring to FIG. 5, shown is a further block diagram illustrating exemplary sub components of an exemplary power control module 201 for controlling the bi-colour LED treatment unit 102. The power control module 201 comprises a port 209 with a power interface 562 to provide power to a power management module 530, and a communication interface 561. The power management module 530 comprises a DC to DC converter. The power management module 530 may alternately, or in addition, comprise an AC to DC converter, or an equivalent circuit to increase or decrease power to a selected level for one or more light emitting elements for receiving AC power.

The power management module 530 is operable to power a dynamic voltage controller (DVC) 450, which is operable to apply a driving voltage to first, second, and nth LED light emitting elements 118, 120, and 122 in the light source 103 via the switch 440.

The interface 209 also includes a communication connection 564, which enables the treatment head control module 104 to communicate with a controller 542 on the power control module 201. The controller 542 may comprise a microcontroller, FPGA, or other processing circuit and is linked to the switch 440 and operable to actuate the switch 440.

For example, in a bi-colour light source 103 comprising only a first light emitting element 118 and a second light emitting element 120, the controller 542 is operable to actuate the switch 440 to selectively cause the first and second light emitting elements 118 and 120 to emit.

In one example, the switch 440 is a polarity switch which is operable to toggle between a forward and a reverse polarity and the first and second light emitting elements 118 and 120 comprise a bi-colour LED. As such, when the switch 440 is actuated to cause the bi-colour LED to alternate between a forward and a reverse polarity, the colour of emission from the bi-colour LED light source 103 is switched from a first wavelength emitted by the first light emitting element 118 to a second wavelength emitted by the second light emitting element 120.

Specifically, the first LED light emitting element 118 is operable to emit light at a first wavelength when driven with a forward current whereas the second LED light emitting element 120 is operable to emit light at a second wavelength when driven with a reverse current. The first LED light emitting element 118 may be combined with the second LED light emitting element 120 to form a common bi-colour LED array. Such an array enables the light emission from the first LED light emitting element 118 and second LED light emitting element 120 to be substantially uniformly distributed across the surface of the bi-colour LED light source 103.

Bias circuits 522 and 526 are provided to bias the first and a second LED light emitting elements 118 and 120 in their respective operational regimes. Additional bias circuits 529 may be provided to bias n additional light emitting elements 122. Power adjustment modules 520, 524, and 528 associated with each of the light emitting elements 118, 120, and 122, respectively, are operable to receive an input waveform via the command signal 561 from the treatment head control module 104 and provide the input waveform to the polarity switch 440 to cause each colour of bi-colour LED array to be emitted in accordance with the input waveform. For example, the power adjustment modules 520, 524, 528 may enable respective ones of the bi-colour LED array to emit in a modulated, sinusoidal waveform and at a selected duty cycle.

Each of the light emitting elements 118, 120, and 122 is in communication with a current controller 552. The current controller 552 serves as a current source and is operable to control the current applied to the light emitting elements 118, 120, and 122 based on an input from the treatment head control module 104. For example, the current may be selected depending on the current requirements of a particular light emitting element.

The current controller 552 interfaces with LED hardware monitors 558, 559, and 560 associated with each of the LED light emitting elements 118, 120, 122, respectively. The LED hardware monitors 558, 559, and 560 monitor the current flow through arrays of the LED light emitting elements 118, 120, and 122, respectively. For a given intensity of optical output, the current controller 552 maintains the current being driven through each of the LED light emitting elements 118, 120, and 122 substantially constant at a selected current. The current at which an LED light emitting element is driven depends on the power requirements of the LED light emitting element and the desired optical output.

The DVC 450 is operable to dynamically adjust the voltage applied to the LED light emitting elements 118, 120, 122 based on a reading of a voltage in the current controller 552. For example, the DVC 450 may receive feedback from a voltage splitter on the current controller 552 and dynamically adjust the voltage being applied such that the applied voltage is higher than a lower voltage threshold and lower than an upper voltage threshold. The lower voltage threshold is selected to be at, or above, the voltage level required to drive the LED light emitting elements 118, 120, and 122. Maintaining the voltage above a lower voltage threshold enables the LED light emitting elements 118, 120, 122 to emit. Maintaining the voltage below an upper threshold prevents the DVC 450 from applying an unnecessarily high driving voltage, which can lead to excess heat generation within the driving circuitry. The current controller 552 therefore prevents an excess voltage from being applied by the DVC 450 by providing a voltage reading to the DVC 450, which enables the DVC 450 to dynamically adjust the driving voltage such that the driving voltage falls within a selected range.

Example driving voltages of LED light emitting elements may range from about 1 volt up to 3.5 volts or more. For example, an LED light emitting element which emits at 660 nm may require 2.1 to 2.3 volts whereas an LED light emitting element which emits at 840 nm may require only 1.5 to 1.7 volts.

Upon the DVC 450 detecting a voltage, via the current controller 552, that is above a selected threshold, the DVC 450 reduces the driving voltage being applied to the light source 103.

An LED status monitor 441 monitors other parameters of the LED light emitting elements such as temperature, or whether one of the LED light emitting elements is experiencing an electrical fault such as a short. The current controller 552 obtains the current, and any other available parameters, from the hardware monitors 558, 559, 560, and 441 and may adjust the current accordingly.

As mentioned above, the voltage required to drive an LED light emitting element in an LED array depends on parameters associated with that LED array. Specifically, power requirements of an LED light emitting element that emits at a first wavelength may be different from the power requirements of an LED light emitting element that emits at a second wavelength. For example, an LED array emitting at 660 nm may require a 22 volt applied voltage to achieve a particular current whereas an LED array emitting at 830 nm may require a 15 volt applied voltage to achieve the same current. As such, if the applied voltage is not reduced from approximately 22 volts to approximately 15 volts when switching a bi-colour LED light source 103 from a 660 nm LED array to the 830 nm LED array, although the LED array current is kept constant, the excess voltage creates significant heat in the drive circuit, which may eventually be a discomfort or even burn hazard to the patient and may also damage the light treatment system 100. Therefore, when the light source 103 is switched by switch 440 from a higher voltage LED array to a lower voltage LED array, the DVC 450 detects that the voltage across the drive circuit is above a selected threshold and the DVC 450 reduces the driving voltage of the array to reduce the waste heat produced by the drive circuit.

As such, the simplified circuit provided in FIG. 5 compensates for the voltage requirements of various LEDs when switching between emission colours of a bi-colour LED array or a multi-colour LED array to maintain the current below a selected threshold or within a selected range. For example, to generate approximately 1000 mW of optical output at 660 nm, a current of approximately 180 mA must be applied to the array of red LED light emitting elements. To generate approximately 2000 mW of optical output at 840 nm, a current of approximately 400 mA must be applied to the array of infrared LED light emitting elements.

Although the power control module 201 of FIG. 5 is explained in the context of using a bi-colour LED array as a light source 103, it will be appreciated that multi-colour arrays may also be used. For example, the LED array may comprise three or more colours, in which case, the switch is operable to select between the three LED colours, or between combinations of these three colours. Upon the DVC 450 determining, via the current controller 552, that the voltage has exceeded a selected voltage threshold, the DVC 450 reduces the driving voltage, thereby maintaining the driving voltage below the selected threshold.

As will be appreciated, the voltage applied to an LED must be higher than a characteristic threshold voltage to cause an LED to emit. It will be appreciated that biasing circuits 522, 526, and 529 are used maintain each of the LED light emitting elements 118, 120, and 122 above their respective threshold voltages. For example, biasing circuits 522, 526, and 529 may apply a voltage to an LED that, when combined with a signal voltage, produces a driving voltage that is above the threshold voltage of the LED.

The biasing circuits 522, 526, and 529 may, for example, be operable to produce a biasing voltage that is at or slightly higher than the threshold voltage of the LED. If the threshold voltage of a first LED of a bi-colour LED is 1.9 volts whereas the second LED of the bi-colour LED is 1.8 volts in the reverse polarity, biasing circuit 522 may apply a 1.9 volt bias in the forward direction when the switch 440 is in the forward position whereas biasing circuit 526 may apply a 1.8 volt bias in the reverse direction when the switch 440 is in the reverse position. As such, the voltage applied by the DVC 450 to drive the LED can be below the threshold voltage before being biased by the biasing circuits 522 and 526.

This bias enables the LED light emitting elements 118 and 120 to illuminate substantially immediately following the actuation of the switch 440 and ensures that the LEDs are driven with a voltage above the minimum threshold. Moreover, the bias assists the power control module 201 in linearly controlling the optical output of the LED light emitting elements 118 and 120 as the current is varied.

Referring now to the simplified diagram of FIG. 4, the power control module 201 may further drive additional light emitting elements 122, having n different wavelengths, respectively. It will be appreciated that two or more light emitting elements may also share the same wavelength. FIG. 4 is a simplified example of a multi-colour treatment head 102 operable to select among up to n light emitting elements 118, 120, and 122 emitting at λ1, λ2, and λn, respectively. The DVC 450 located in the power control module supplies a voltage to each of the light emitting elements 118 120, and 122. Switch 440, located between the DVC 450 and the light emitting elements 118, 120, and 122 is operable to switch between each of the light emitting elements 118, 120, and 122.

Although the invention has been described with reference to certain specific embodiments, various modifications thereof will be apparent to those skilled in the art without departing from the invention as outlined in the claims. The entire disclosures of all references recited above are incorporated herein by reference.

What is claimed is:

1. A flexible light treatment pad comprising:
one or more light source modules interconnected in a continuous electrical web and disposed on a flexible circuit board located on a first portion of the light treatment pad, and
a control module located on a second portion of the light treatment pad, the control module for providing a control signal to the one or more light source modules,
wherein said light source modules and said control module are encased in a flexible moulding layer.

2. The flexible light treatment pad of claim 1, wherein a discontinuity is provided between the first portion and the second portion to reduce the mechanical connection between the portions.

3. The flexible light treatment pad of claim 2, wherein said discontinuity extends partially across the flexible treatment pad for providing a physical separation between the first and the second portion and delimit a bridge configured to allow electrical connectivity between the first and the second portion.

4. The flexible light treatment pad of claim 1, wherein each light source module comprises a light emitting diode (LED) electrically connected between two connector pad terminals, the connector pad terminals providing a heat sink body.

5. The flexible treatment pad of claim 4 wherein said connector pad terminals are disposed on the opposite side of said board to said LED's

6. The flexible treatment pad of claim 5 wherein said connector pad terminals collectively extend over a substantial portion of said opposite side.

7. The flexible light treatment pad of claim 1, wherein the flexible moulding layer is optically clear and disposed surrounding said control module and the light source, the moulding layer selected from the group consisting of: Silicone, thermoplastic, polyurethane, and thermoplastic elastomer (TPE).

8. The flexible light treatment pad of claim 7, wherein the flexible moulding layer comprises two transparent Silicone layers encasing said light source modules and the control module.

9. The flexible light treatment pad of claim 1, wherein the flexible moulding layer is configured to retain shape of a treatment surface of a patient for subsequent application.

10. The flexible light treatment pad of claim 1, wherein the control module further comprises a connector interface for connecting with an external user interface for controlling operation of said one or more light source modules.

11. The flexible light treatment pad of claim 1, wherein each light source module comprising light emitting elements comprising LASERs.

12. The flexible light treatment pad of claim 3, wherein the control module and the bridge are substantially inflexible along a first axis, such that the treatment pad can be shaped and flexed concavely or convexly along a second axis transverse to the first axis.

13. The flexible light treatment pad of claim 1 wherein a barrier is interposed between said circuit board and said housing.

14. The flexible treatment pad of claim 13 wherein said barrier is a non-woven web.

15. The flexible treatment pad of claim 14 wherein said housing is formed as a pocket having a peripheral edge to locate said board in said pocket.

16. The flexible treatment pad of claim 15 wherein said board is retained in said pocket by an optically clear layer connected to said housing.

17. A method for manufacturing a flexible light treatment pad, the method comprising:
a. rolling first and second moulding layers to respective pre-defined thicknesses;
b. cutting said moulding layers to a pre-defined size;
c. layering said first and second moulding layers to encase a generally flexible circuit board, the circuit board comprising one or more light source modules interconnected in a continuous electrical web disposed on a first portion of the circuit board and a control module located on a second portion of the circuit board; and,
d. compression moulding and volcanizing said layers and the flexible circuit board to form the flexible light treatment pad.

18. The method of claim 17, wherein said pre-defined thickness and size is defined to allow flexing and movement of the light treatment pad.

19. The method of claim 17, wherein the second portion is physically separated and spaced apart from the first portion.

20. The method of claim 17 wherein said first moulding layer disposed on a top surface of the light source module is thinner than the second moulding layer disposed on a bottom layer of the light source module, the second moulding layer configured for contact with a surface area to be treated.

21. The method of claim 17, wherein said first and second moulding layers are made from a material selected from the group consisting of: rubber, silicone, thermoplastic elastomers, PVC and/or polyurethane.

22. The method of claim 17 further comprising: embedding a shape memory alloy within the flexible light treatment pad for retaining the shape of contours of the surface area being treated.

23. A method of manufacturing a flexible light treatment head having an array of light source modules disposed on a circuit board comprising the steps of forming a flexible housing with a pocket defined by a peripheral edge, locating the board within the pocket, and securing the board within the pocket by applying a layer of material across the board.

24. The method of claim 23 wherein said layer is over molded to connect to said housing.

25. The method of claim 24 wherein said board is disposed in said pocket with light emitting sources of said modules exposed and said layer is disposed over said sources.

26. The method of claim 25 wherein said layer is an optically clear material.

27. The method of claim 23 including the step of placing a barrier in said pocket prior to location of said board.

28. The method of claim 27 wherein said barrier is a non-woven web.

29. The method of claim 27 including the step of locating a barrier over said board prior to application of said layer.

30. The method of claim 29 wherein said barrier is non-woven.
